(19) United States
(12) Patent Application Publication
(10) Pub. No.: US 2004/0124108 A1
(43) Pub. Date: Jul. 1, 2004

(54) Passive method of high vapor pressure stabilization

(75) Inventor: Igor V. Touzov, Cary, NC (US)

Correspondence Address: IGOR V TOUZOV, 311 CASTLE HAYNE DRIVE, CARY, NC, 27519, US

(73) Assignee: Igor V Touzov, Cary, NC (US)

(21) Appl. No.: 10/707,633

(22) Filed: Dec. 26, 2003

Related U.S. Application Data
(60) Provisional application No. 60/319,816, filed on Dec. 27, 2002.

(51) Int. Cl. A61B 19/02

(52) U.S. Cl. 206/438

(57) ABSTRACT

Methods of passive stabilization and control of relative vapor pressure at nearly saturated conditions. These methods and devices with their use are suitable for short and long term containment of microfluidic systems, live tissues, organs, plants, grocery products. Devices are extremely low cost and versatile.

### CROSS REFERENCE TO RELATED APPLICATIONS

This application is a regular application of provisional Patent Application No. 60/319,816, filed Dec. 27, 2002 which is hereby incorporated by reference in its entirety for all purposes.

### BACKGROUND OF INVENTION

The present invention relates to the field of producing humidified gas environment with precisely stabilized relative vapor pressure at nearly saturated conditions.

Humidified gases such as argon, nitrogen, air, oxygen, hydrogen, carbon dioxide, helium and various mixes are widely employed by numerous industries. Subset of their applications comprises storage and incubation operations, wherein some substances exposed to nearly static environment for extended time period that ranges from seconds to years. The term storage comprises nearly all conditions when objects or substances undergo stationary exposure to gaseous environment. Some examples of these conditions are storage of fruits and vegetables, preservation and transport of live human tissues and organs.

In resent years, many technological processes featuring microfluidics and other novel techniques become dependent on accurate control of composition of gaseous environment that contains nearly saturated vapors of water or other chemicals. Most of these processes currently rely on active humidity control means such as humidistat, etc.

Many related technologies such as biological micro-arraying employs jet based dispensing equipment to produce patterned surfaces. Some of these techniques utilize intermediate steps of drying and sequential dissolving of patterned chemicals, others use purging of microchannels to remove dried substances (process similar to clean-up cycle of typical inkjet printer head). All these conditions are caused by inaccurate control of vapor pressure.

It is inherently difficult to stabilize open surface of microscopic amount of liquid due to high evaporation rates caused by significant surface curvatures. The only stable environmental conditions for such liquids are nearly saturated or event oversaturated vapor environments. Due to known instability of oversaturated vapors it is impossible to create passive system that can maintain such conditions for reasonably long time period. Due to natural events such as small fluctuation in pressure and temperature, as well as uncontrolled flow of atomic and subatomic particles, oversaturated vapors tend to precipitate on contacting surfaces. Such conditions cause undesirable effects on stored devices, substances, or other items.

In most cases open surfaces of liquids have negative integral curvature that allows successful stabilization using nearly saturated vapors. While theoretically it is possible to create a passive device that can maintain nearly saturated vapor environment for an extended time period, in practice construction of such device meets aforementioned instability problems. Nearly saturated vapors can easily reach saturation point due to changes in pressure and temperature. This in turn causes undesirable condensation of liquid on exposed surfaces, resulting in uncontrolled surface processes and possible damage of surface structures.

Based on the above discussion, it is clear that there is a need for a system capable of passive stabilization of nearly saturated vapors. Benefit of passive approach is ability of long term storage that does not depend on power sources or measurement techniques.

### SUMMARY OF INVENTION

The present invention presents method and devices for passive control and stabilization of vapors of chemicals at saturated and nearly saturated conditions. The method of invention relies on neither power source nor sensors. It utilizes chemical and physical characteristics of materials used in constriction of physical device as well as geometry of said device to eliminate any need of active control or monitoring of composition of gaseous environment.

The method of the invention uses at least two distinct porous substances to control relative pressure of specific vapors. This condition sets fundamental distinction between the present invention and all prior art. Prior art publications and inventions actively employ porous materials to deliver liquids or chemicals to gaseous environment for purposes of creation of controlled or uncontrolled concentration of desirable chemicals in the gas.

The method of present invention uses substances with large surface area, wherein at one moment of time said materials functions as a source of vapors while in another moment they operate as absorbents and thus remove excessive amount of vapors.

### BRIEF DESCRIPTION OF DRAWINGS

FIG. 1 is schematic view of device with two distinct absorbing surfaces constraining lowest and highest vapor pressure.

FIG. 2 is crossection of sealed enclosure with stabilized vapor content.

FIG. 3 is large scale full or partial enclosure with stabilized vapor composition.

FIG. 4 is diagram showing two implementations for partially open enclosures.

### DETAILED DESCRIPTION

All methods and apparatuses described in this section of the document may be further extended to include additional parts and steps. These steps include, but not limited to, sterilization by gamma or X-rays, preservation by adding preservatives into liquids or vapors or gases, thermal cycling, etc.

Definitions

Terms containment apparatus and apparatus for containment are defined as an apparatus or enclosure that provides required environmental conditions for objects that are inside of their work area or volume.

Packaging Method with Semi-Saturated Porous Materials

The method of the invention uses porous material or materials with large surface area to hold liquid chemicals on said surfaces or in the pores. The material(s) composition is selected to have high affinity to the liquid. Liquid is loaded into the material either by exposure to chemicals at vapor/gas state or by soaking the material in the liquid and evaporating excessive amount of the liquid content. Said material(s) is(are) enclosed into sealed volume that also contains a subject of containment operation.

This method allows stabilization of partial pressure of the vapors in the volume containing aforementioned porous material(s). Specific size of said pores or chemical properties of said surfaces is achieved by selection of the material or additional chemical processing of its surface. Affinity of the porous material to the liquid causes negative curvature of the liquid's meniscus inside the pores, or causes formation of chemisorbed layer of the liquid on said surfaces. This defines partial pressure of vapors of the liquid in equilibrium state. The method of the invention allows achieving stable vapor pressure at any predetermined range of values by selection of appropriate porous material and/or surface treatment of selected surfaces. Selection of porous materials with assorted or variable sizes of the pores allows using such material for different vapor pressure ranges by adjusting volume of deposited liquid.

In some cases the gels incorporating desired chemicals can be uses as a substance with said high surface area. The drawback of use of the gels as a porous substance is instability of their geometrical properties that might in some cases limit their operational time due to irreversible changes in their geometry.

Fundamental advantage of this method is ability to stabilize vapor pressure at vicinity of 100% of its relative pressure. As an example, selection of silica gel material with 100-micron pore size and saturation of it with water vapors allows achieving stable humidity at level 99.9989%.

Amount of the liquid loaded into the material does not have to saturate it.

Reduction of temperature of the vapors causes transfer of chemicals of the vapors from vapor volume to the surface of said porous material(s). Increase in temperature causes an opposite effect.

The method of invention provides efficient and cost effective way for handling, storage and transportation of substances or items that require high partial pressure of vapors. The areas of uses comprise, but are not limited to, bio arrays, micro fluidic devices, biological tissues, grocery produce, etc.

Packaging Method with Heterogeneous Porous Materials

The method of the invention uses at least two types of porous materials. First type is loaded with liquid as described in previous embodiment. Second type remains unloaded. Materials are enclosed into sealed volume that also contains a subject of containment operation.

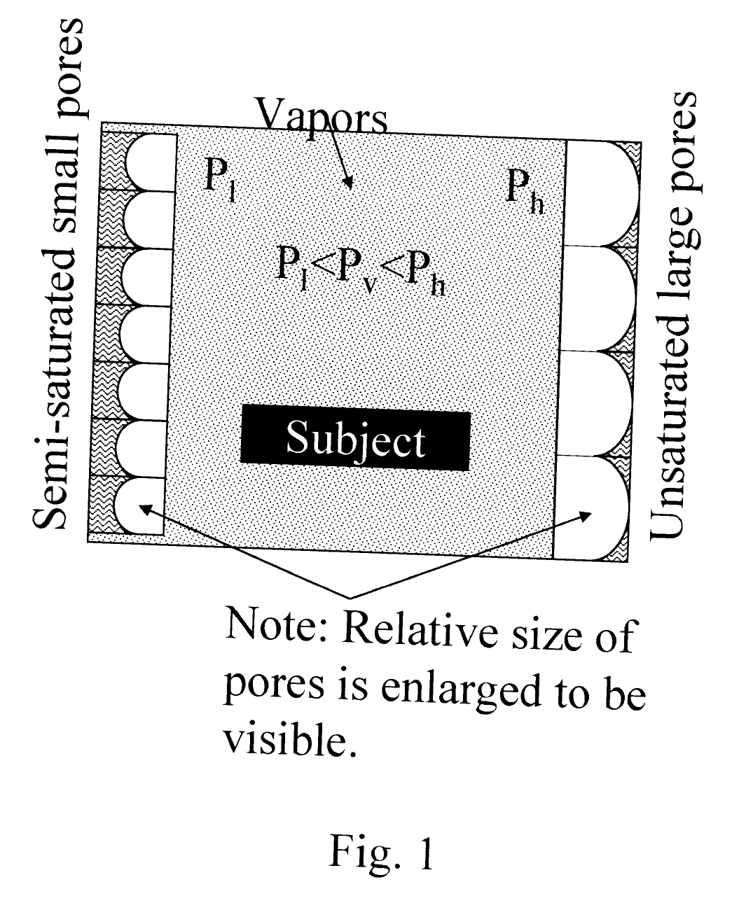

This method operates on the same principal as the invention described in the previous embodiment. The addition of second type of the porous material allows preventing of undesirable condensation of the vapors on surfaces of the subject of packaging operation. Size of pores or affinity to the liquid of the second type of material is selected in a way that equilibrium vapor pressure over the surface of said first material is less then over the surface of said second material as illustrated on FIG. 1. A rapid change of temperature of the vapor might cause an increase in partial vapor pressure. The second material is capable of quick absorption of the vapors before their concentration reaches 100% (saturated state), and thus prevents undesirable condensation.

This method may be substituted by the method described in previous embodiment, which employs the use of porous material with heterogeneous size of the pores, or heterogeneous surface properties of the material. This effect may be achieved by mixing particles of several porous materials together.

The methods described in current and previous embodiment may be extended to the use of multiple porous materials containing different liquids.

Applications of this method comprise, but are not limited to, all applications described in the previous embodiment. This method provides higher stability during rapid temperature or pressure changes, and eliminates condensation of vapors on surfaces inside the package.

As an example the method can be implemented using silica gel particles with effective pore sizes 10 micron and 100 micron. Particles with smaller pore sizes are soaked in water and placed in the same enclosed volume as particles with larger pores. Physical separation between these two groups prevents surface-to-surface transfer of the liquid between these two groups. Effective vapor pressure or humidity inside said enclosure will be 99.90%. These conditions will remain stable when temperature or pressure in the enclosure gradually changes. In event of rapid temperature drop or pressure increase the relative vapor pressure become higher and excessive amount of liquid adsorbs on particles with larger pores size, thus limiting vapors pressure to 99.9989%, which prevents their condensation.

Autonomous Passive Containment Apparatus

Apparatus for long- or short-term storage consists of, but not limited to, porous materials, subject of containment, subject holder, enclosure, and may include particle air filter.

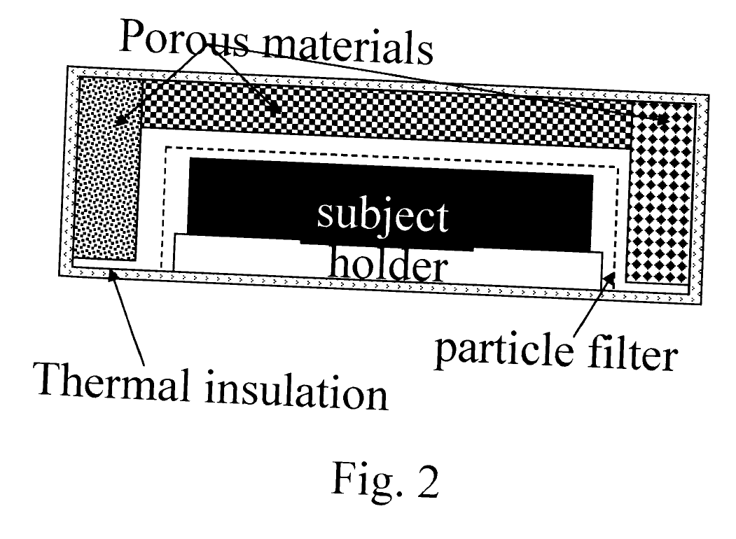

FIG. 2 illustrates a variant of the apparatus's design. It consists of subject of storage that is mounted on holder element that ensures proper position of the subject during storage. The subject may be sealed from particle contamination by particle filter element(s) that is transparent to vapors. Various porous materials may be mounted inside the volume of the enclosure. These materials are prepared according to the methods disclosed in this document. Thermal insulation layer, and/or reflective layer may be used to protect the enclosure from rapid temperature changes and radiation fluxes. Foam materials, Dewar, or thermos are examples that may be used as the thermal insulation layer.

Containment Apparatus with Power Source

Apparatus for long- or short-term storage consists of, but not limited to, porous materials, subjects of containment, thermal elements, full or partial enclosure, and may include air circulation device. In case of partial enclosure the subject of storage is locates in direct proximity of porous materials, or downstream from the porous materials in path of airflow generated by air circulation device.

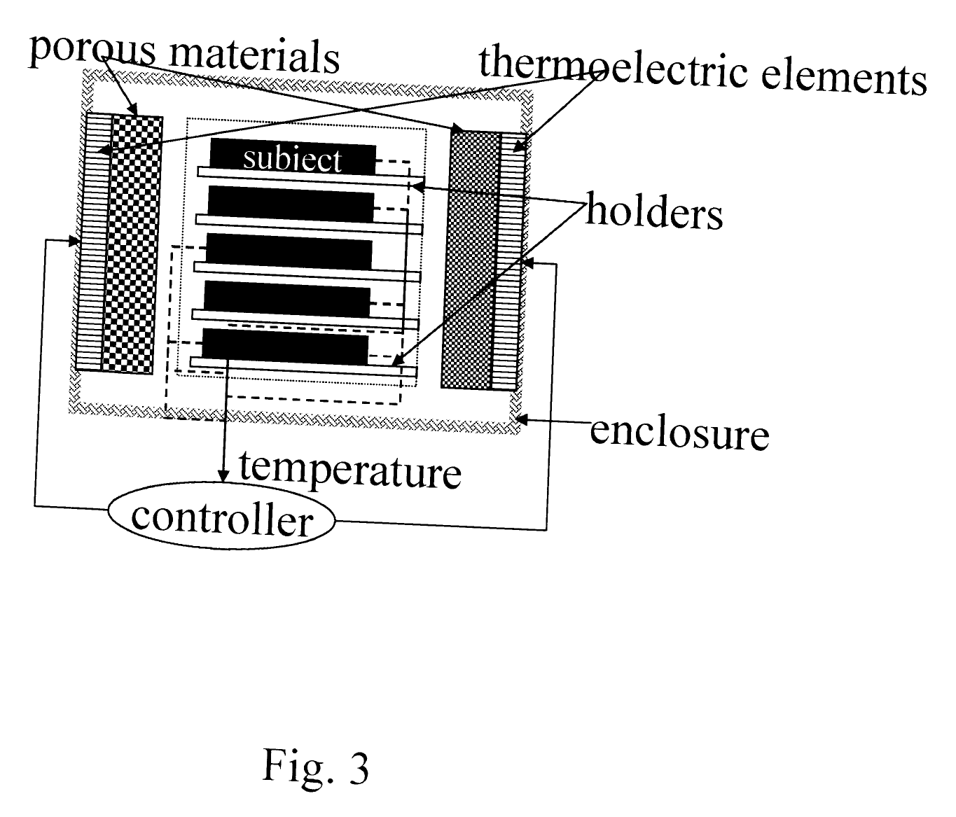

FIG. 3 illustrates a variant of the apparatus that uses complete enclosure.

The subjects of storage are mounted on holders, which ensures their proper position. Holders may perform additional functions that may comprise, but not limited to, thermal stabilization, shaking, vibration insulation, etc. Apparatus has passive or active particle air filtration system. Some porous materials are mounted on thermal elements that may be used to adjust the temperature of these materials, and as well may be used to monitor temperature of them and heat flux through the thermal elements.

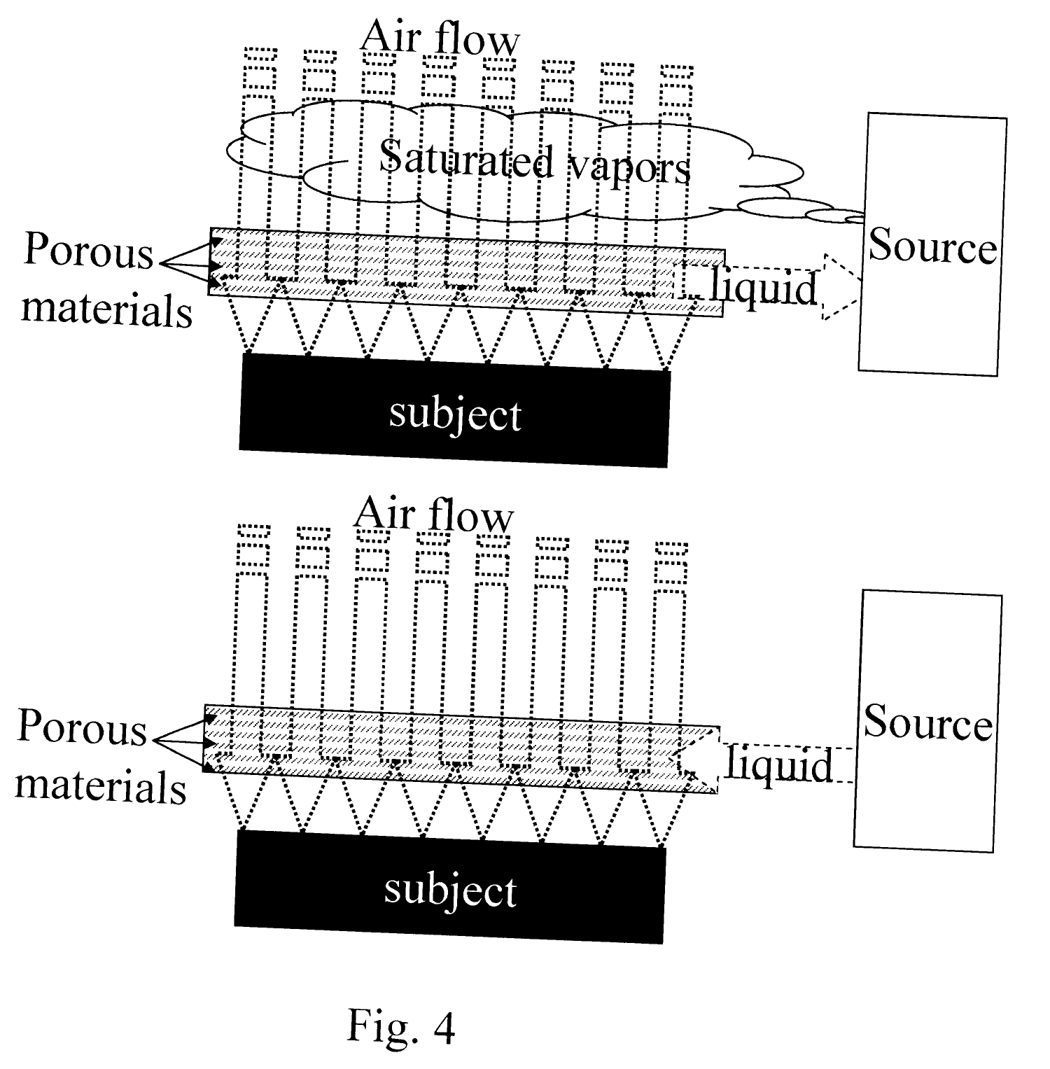

Apparatus may include reservoir or other source of the liquids. In case of partial enclosure this source delivers controlled amount of liquid or saturated vapors toward the porous material as it is illustrated on FIG. 4. Partial pressure of vapors reaching the subject is defined by composition of the porous materials and may be adjusted by temperature of said materials. Apparatus may also contain shutter mechanism that redirects airflow to pass through different areas of said materials or different porous materials.

Upper part of FIG. 4 shows the apparatus that uses porous or high surface area materials to remove excessive amount of vapors from passing gaseous phase. Source of said liquid creates saturated or slightly oversaturated vapors. After passing through said porous substance the vapors pressure is adjusted in accordance with the second method of the present invention. Adsorbed fluid is then returned back to the reservoir or disposed.

Lower portion of FIG. 4 uses porous materials in accordance with the first method of the present invention. Liquid is delivered to the porous substance from reservoir and passing gas is mixed with its vapors. Since the apparatus can be partially open then some losses of vapors occur into ambient environment. Construction of the apparatus designed to provide sufficient surface area of said porous substance to account for such losses.

The distinction of this apparatus from one discussed in the previous embodiment is its ability to enclose larger volumes, wherein uniform composition of gaseous environment can not be sustained by means of passive diffusion of vapors. Addition of active air circulation and temperature controller allows compensation for thermal gradients in ambient environment. Additional liquid source accounts for possible or designed losses of the vapors into ambient environment.

1. A method of containment or enclosure that employs one or all of the following substances: porous materials; gels; materials with large surface area; porous particles. And wherein at least one of said substances contains vapor generating chemical(s) or liquid(s) contacting its surface by means of at least one of the following:
adsorption; chemisorptions; capillary forces.

2. Containment apparatus that uses method of claim 1.

3. Method of claim 1 used for packaging of items during their transportation by air, sea, or ground.

4. Method of claim 1 that uses two or more distinctive types of said substances.

5. Containment apparatus that employs method of claim 4.

6. Apparatus for long- or short-term containment that does not have power source and maintains high partial vapor pressure at predetermined level above 98%.

7. Containment apparatus that uses power source and maintains relative pressure of vapors at 98% or above and has a source of saturated or oversaturated vapors.

8. Containment apparatus of the claim 7 that is used for packaging and storage of micro fluidic devices or materials.

9. Containment apparatus of the claim 7 that is used for transportation of items by air, sea, or ground.

10. Apparatus of the claim 6 that is used for transportation of items by air, sea, or ground.

11. Method of the claim 4 used for packaging of items during their transportation by air, sea, or ground.

12. Apparatus that controls vapor pressure near saturation level and uses meniscuses curvatures and temperature of fluid to adjust said pressure.

13. Use of the apparatus of claim 12 as a part of enclosure for MEMS device.
